(19) United States
(12) Patent Application Publication
(10) Pub. No.: US 2008/0188749 A1
(43) Pub. Date: Aug. 7, 2008

(54) Three Dimensional Imaging for Guiding Interventional Medical Devices in a Body Volume

(75) Inventors: Volker Rasche, Erbach (DE); Raoul Florent, Ville d'Avray (FR); Olivier Gerard, Viroflay (FR)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., EINDHOVEN (NL)

(21) Appl. No.: 11/910,620

(22) PCT Filed: Apr. 4, 2006
(86) PCT No.: PCT/IB2006/051039

§ 371 Date: Oct. 4, 2007

(30) Foreign Application Priority Data
Apr. 11, 2005 (EP) 05300272.1

(51) Int. Cl. A61B 8/00

(52) U.S. Cl. 600/443

(57) ABSTRACT

The present invention relates to an intervention guidance system in which the location of an interventional medical device (30) within a body volume is determined by image processing means (14) from live, three-dimensional ultrsound images thereof, and this localisation is used to generate a control signal for steering an ultrasound beam (20) so as to alter the imaging plane (or region of interest 235) in accordance with the relative location within the body volume of the intervention device (30). The use of image processing techniques to localise the intervention device (30) obviates the need for specific locations on the device.

The invention relates generally to three dimensional diagnostic imaging and, more particularly, to the use of three dimensional ultrasonic diagnostic imaging to guide the placement and/or operation of invasive (interventional) medical devices within a body volume.

### BACKGROUND OF THE INVENTION

Ultrasonic imaging is commonly used to image the insertion, use or operation of medical devices and instruments within the body. For example, the growing interest in minimal-invasive methods for treatment of cardiac diseases necessitates the development of methods and devices allowing the physician to guide a medical instrument to predetermined positions inside or outside the heart. In electrophysiology, for example, it is necessary to guide a catheter to a plurality of predetermined positions in the ventrical or atrial walls in order to measure an electrical pulse or bum wall tissues.

U.S. Pat. No. 6,587,709 discloses a system for guiding a medical instrument in the body of a patient. Such a system acquires a live 3D ultrasound image data set using an ultrasound probe. An advantage of acquiring a 3D image data set is to obtain depth information. An advantage of using a live 3D ultrasound image modality is that the surrounding anatomy is visible, which facilitates the guidance of the medical instrument by the physician. The system further comprises localisation means for localising the medical instrument within the 3D ultrasound data set, which locates three ultrasound receivers mounted on the medical instrument relatively to the ultrasound probe. Such localisation allows for automatic selection of a plane to be imaged, which comprises at least a section of the medical instrument. Therefore, no readjustment of the ultrasound probe position by hand is necessary in order to track the progress of the medical instrument within the body volume.

However, the system described in U.S. Pat. No. 6,587,709 requires the use of a dedicated catheter (or other medical device), in the sense that ultrasound receivers are required to be provided on the catheter. These receivers are capable of detecting the ultrasound pulses that are generated by the ultrasound system, and an image processing system then calculates in real time the position of the receivers such that they, and therefore the catheter, can be localised relative to the ultrasound transducer that is situated outside the body. The image processing unit then uses the known positions of the ultrasound receivers to select a suitable imaging plane from the volumetric ultrasound data so as to display this plane on a monitor.

### SUMMARY OF THE INVENTION

It is an object of the present invention to provide an image processing system and method of localising an interventional medical device or other selected reference feature relative to a body volume so as to enable a suitable imaging plane to be selected for display, whereby no dedicated sensors or receivers are required to be provided in or on the reference device such that the system can be used, without modification, in several different 3D medical imaging applications.

In accordance with the present invention, there is provided an imaging system for generating for display live, three-dimensional images of a body volume, the system comprising scanning means for scanning said body volume so as to obtain three-dimensional image data in respect of said body volume, object recognition means for identifying, within one or more of said live images of said body volume, the relative location of a selected object within said body volume, means for selecting an imaging plane corresponding to said location of said object, and means for generating a control signal for steering said scanning means relative to said body volume so as to obtain three-dimensional image data in respect of said selected imaging plane.

Thus, once the imaging plane has been selected in accordance with the localisation of the selected object within the body volume, a control signal is generated to automatically steer the scanning means relative to the body volume so as to obtain three-dimensional image data representative of the body volume in respect of the selected imaging plane. The control signal may be arranged to electronically steer an incident beam, while the scanning means or probe from which it emanates remains stationary relative to the body volume. Alternatively, the control signal may be arranged to mechanically steer the probe itself to achieve the selected imaging plane.

A significant advantage of the system of the present invention is that it does not require a specific medical instrument, such as a medical instrument equipped with active localisers. Considering the fact that the medical instrument needs to be changed for each new patient, the resultant cost savings are significant.

The location of the selected object, which may be a medical intervention device or an anatomical landmark, may be determined by segmenting or filtering said live images to enhance the appearance therein of said selected object, and then defining the location of the object within the body volume by one or more reference points relative to at least a portion of the object. Means are preferably also provided for determining the orientation of the object relative to the body volume.

In one exemplary embodiment of the present invention, the location and/or orientation of the object may be used to select one or more parameters for visualisation of the body volume, such as the selection of one or more portions of said live images for visualisation, suppression and/or alignment with the object.

The scanning means may comprise means for generating an incident beam and receiving a beam reflected from a transmitter through said body volume so as to obtain three-dimensional image data in respect of the body volume, in which case the control signal is configured to steer the incident beam over the body volume to achieve the selected imaging plane. This is particularly pertinent when the imaging system is, for example, a 3D ultrasound system. However, the present invention is not necessarily intended to be limited to this modality, and other three-dimensional imaging systems, such as MRI or VCT may be used.

These and other aspects of the invention will be apparent from and will be elucidated with reference to the embodiments described hereinafter.

### BRIEF DESCRIPTION OF THE DRAWINGS

The present invention will now be described in more detail, by way of example, with reference to the accompanying drawings, wherein:

FIG. 1 illustrates in block diagram form the use of three dimensional ultrasonic imaging to guide or monitor an invasive instrument or procedure;

FIG. 2 is a schematic drawing of means for use in an exemplary embodiment of the present invention, for localising the medical instrument and determining an imaging plane comprising the medical instrument within the 3D ultrasound data set;

FIGS. 3 a and b illustrate schematically the principle of region-of-interest adaptation employed in an exemplary embodiment of the present invention, whereby the ultrasound beam is steered electronically; and

FIG. 4 illustrates schematically a system according to an exemplary embodiment of the present invention, whereby the region-of-interest is adapted by mechanical steering of the scanhead.

### DETAILED DESCRIPTION OF THE INVENTION

The present invention provides an imaging system whereby the localisation of an interventional medical device, or other reference object, within a body volume is used to control an imaging device so as to obtain three-dimensional images of the body volume in respect of a selected imaging plane. In the following, the three-dimensional imaging modality referred to will be live 3D ultrasound imaging, but it will be appreciated that the present invention is equally applicable to any other modality that provides real-time volume information, such as, for example, MRI (magnetic resonance imaging) or VCT (volume computerised tomology).

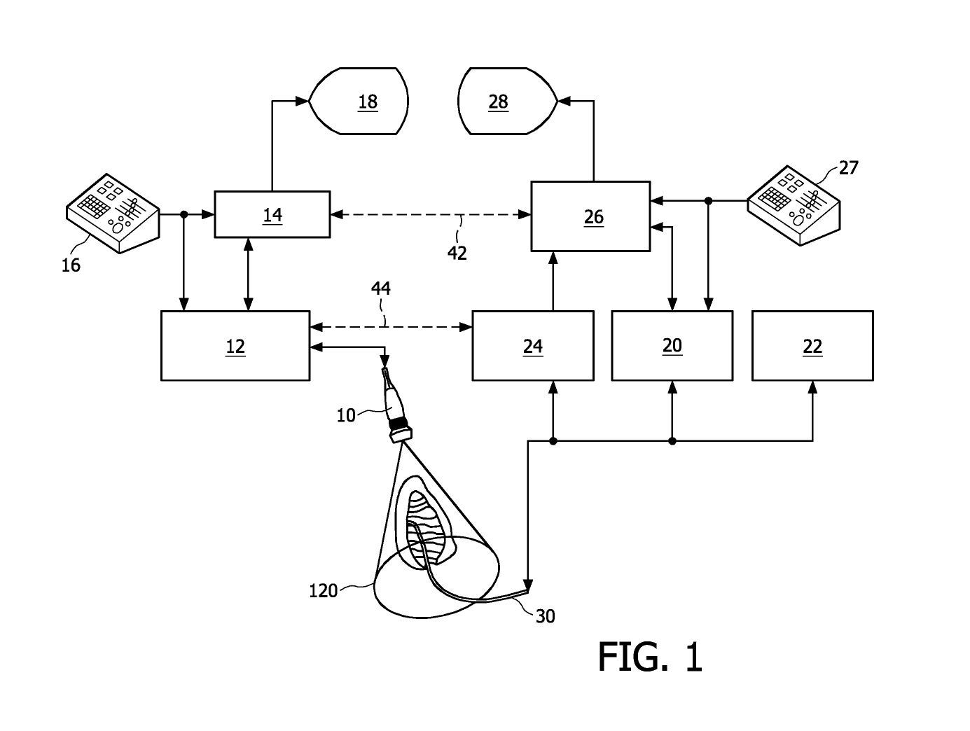

Referring first to FIG. 1 of the drawings, the use of three dimensional ultrasonic imaging to guide or monitor an invasive instrument and procedure is shown in partial block diagram form. On the left side of the drawing is a three dimensional (3D) ultrasonic imaging system including a probe 10 having a two dimensional array transducer. The transducer array trasmits ultrasonic beams over a volumetric field of view 120 under control of an ultrasound acquisition subsystem 12 and receives echoes in response to the transmitted beams which are coupled to and processed by the acquisition subsystem. The echoes received by the elements of the trasnducer array are combined into coherent echo signals by the acquisition subsystem and the echo signals along with the coordinates from which they are received (r, θ, φ for a radial transmission pattern) are coupled to a 3D image processor 14. The 3D image processor processes the echo signals into a three dimensional ultrasonic image which is displayed on a display 18. The ultrasound system is controlled by a control panel 16 by which the user defines the characteristics of the imaging to be performed.

Also shown in FIG. 1 is an interventional device system. The interventional device system includes an invasive (interventional) device 30 which performs a function within the body. In the drawing, the interventional device is shown as a catheter, but it could also be some other tool or instrument, such as a needle, a surgical tool such as a dissection instrument or stapler or a stent delivery, electrophysiology, or balloon catheter, a therapy device such as a high intensity ultrasound probe or a pacemaker or defibrillator lead, a diagnostic or measurement device such as an IVUS or optical catheter or sensor, or any other device which is manipulated and/or operates within the body. The interventional device 30 is manipulated by a guidance subsystem 22 which may mechanically assist the manoevring and placement of the interventional device within the body. The interventional device 30 is operated to perform its desired function such as placing an item at a desired location, or measuring, illuminating, heating, freezing or cutting tissue under the control of an interventional subsystem 20. The interventional subsystem 20 also receives information from the interventional device on the procedure being performed, such as optical or acoustic image information, temperature, electrophysiologic, or other measured information, or information signalling the completion of an invasive operation. Information which is susceptible of processing for display is coupled to a display processor 26. Information pertinent to the functioning or operation of the interventional device is displayed on a display 28. The interventional device system is operated by a user through a control panel 27.

The invasive procedure is assisted by visualising the site of the procedure by use of the three dimensional ultrasound system. As the interventional device 30 is manipulated within the body, the three dimensional environment in which the device is operated can be visualised in three dimensions, thereby enabling the operator to anticipate turns and bends of orifices and vessels in the body and to precisely place the working tip of the interventional device at the desired site of the procedure.

In accordance with this exemplary embodiment of the present invention, the image processor 14 is arranged and configured to determine, from the three dimensional ultrasound images acquired by the ultrasound acquisition subsystem 12, the location within the body volume of the interventional device 30. The location within the body volume of the interventional device 30 determines the best imaging plane from which to visualise the progress of the device 30 and the ultrasound acquisition subsystem 12 includes means for manoevring and repositioning the probe 10 so as to constantly keep the interventional device 30 within the probe's volumetric field of view. In a preferred embodiment, the probe 10 has a two dimensional array which rapidly transmits and receives beams steered electronically based on the determined location of the device 30 within the body volume, rather than a mechanically swept transducer, such that real-time three dimensional ultrasonic imaging can be performed and the interventional device and its procedure can be observed continuously and precisely in three dimensions.

Object recognition and/or tracking within three dimensional images is known, and many different techniques are envisaged to be suitable for use in the present invention, which is not necessarily intended to be limited in this regard. For example, the determination of the lcation of the interventional device within the body volume may be achieved using a filter for enhancing and thresholding elongate shapes.

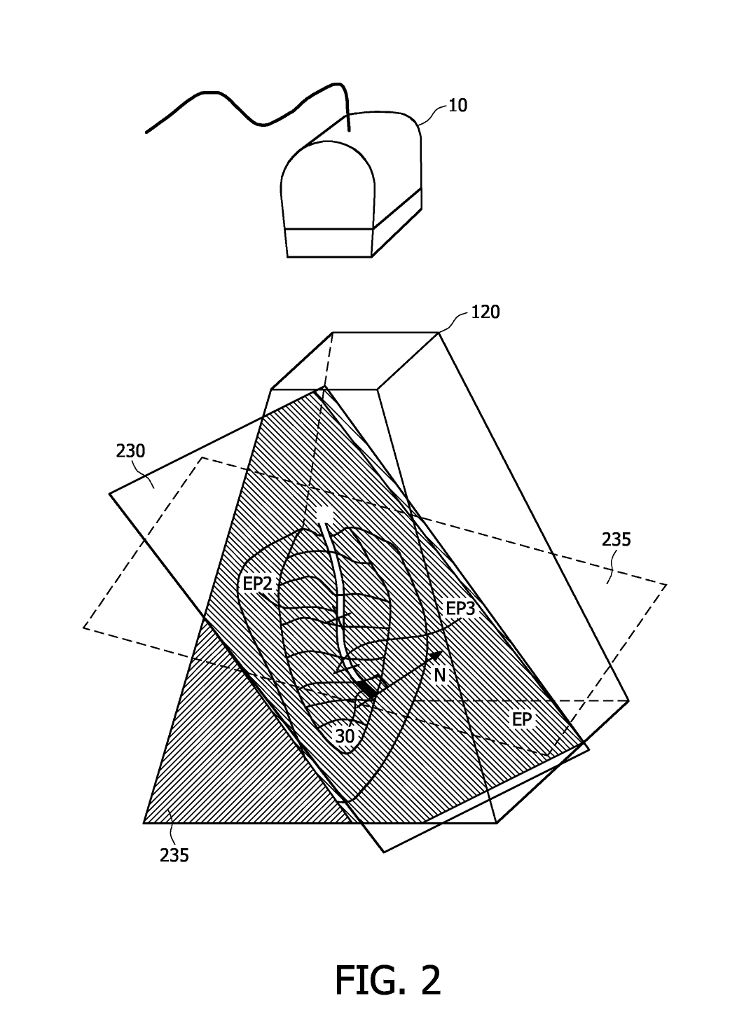

Referring to FIG. 2 of the drawings, the system in accordance with an exemplary embodiment of the present invention comprises means for detecting the position (and orientation) of the medical instrument 30 within the 3D ultrasound data set 120 acquired by the ultrasound acquisition subsystem 12, substantially simultaneously with 3D ultrasound image acquisition. A reference plane comprising a part of the medical instrument 30 is defined and a region of interest (ROI) 235 is obtained, for example by cropping a 3D ultrasound data subset (denoted by the pyrimidal beam 120), which lies behind the reference plane, or by cropping a slab which is formed around the reference plane. In this way, structures that could occlude the visibility of the medical instrument in the 3D ultrasound data set are removed. The region of interest 235 may be user selected or predefined.

It should be noted that the medical instrument often appears with high contrast within the 3D ultrasound data set. It is, for instance, the case of an electrophysiology catheter, which comprises a metal tip at its extremity. The tip is a small, thin segment, which is very echogen and leaves a specific signature in the 3D ultrasound data set. Therefore, either the tip end is considered as a punctual landmark or the whole tip can be considered as an elongate landmark.

Consequently, the detection means involve image processing techniques which are well known to a person skilled in the art, for either enhancing a highly contrasted blob or elongated shape in a relatively uniform background.

The detection means enables a reference plane 30 to be automatically defined by a point EP₁ and a normal orientation N, where the point EP₁ for instance corresponds to the detected extremity of the medical instrument 30, for instance the end of the tip, and the normal orientation N corresponds to the orientation of the device 30.

In an alternative embodiment, a reference plane 233 may be defined by at least three non-aligned points EP₁, EP₂ and EP₃ given by the detection of the medical instrument 30.

The defined reference plane determines the imaging plane in respect of which 3D ultrasound images are to be acquired by the ultrasound acquisition subsystem 12.

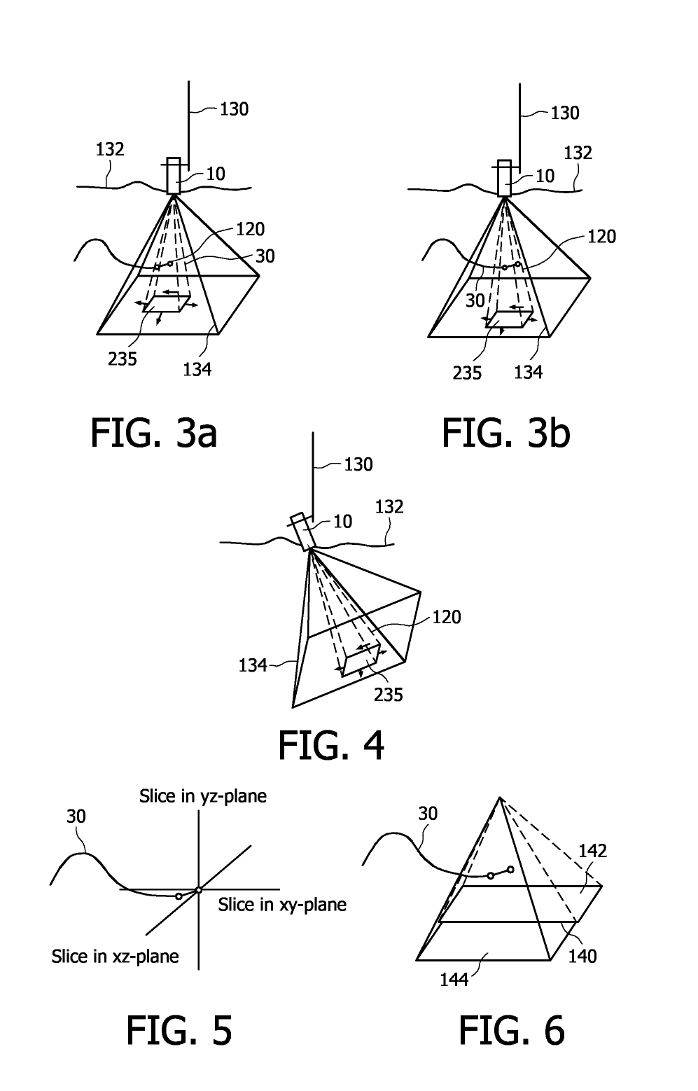

Referring additionally to FIG. 3a of the drawings, the ultrasound acquisition subsystem 12 comprises an ultrasound probe or scanhead 10 mounted on a support 130. The scanhead 10 comprises a two dimensional array transducer. The transducer array trasmits ultrasonic beams over a volumetric field of view 120 under control of an ultrasound acquisition subsystem 12 and receives echoes in response to the transmitted beams which are coupled to and processed by the acquisition subsystem. The echoes received by the elements of the trasnducer array are combined into coherent echo signals by the acquisition subsystem, as explained above.

The location of the medical instrument 30 within the region of interest 235 is determined as described above, and the desired imaging plane is thereby selected. The scanhead 10, which is in contact with the patient's skin 132, may be steered mechanically by for example a dedicated robotic device pressing the scanhead 10 against the patient's skin 132, as illustrated in FIG. 4 of the drawings, so as to alter the orientation of the beam 120 and, therefore, the imaging plane. Alternatively, and as illustrated by FIGS. 3a and 3b, the beam 120 may be steered electronically (with the scanhead 10 in a fixed position against the patient's skin 132) so as to alter the imaging plane according to the location of the medical instrument 30 within the 3D ultrasound data set. Although electronic steering of the beam 120 is thought to be preferable, it is limited to the maximal steering angles of the ultrasound scanhead 10 and hence to a limited volume 134 which can be covered by the device. Thus, if the volume 134 provided by the electronic steering is not sufficient, the scanhead 10 may be mechanically steered to alter the region of interest 235 in accordance with the selected imaging plane.

The region-of-interest adaptation may be performed continuously during movement of the medical intervention device 30 within the body volume, or it can be done in a step-wise manner when movement of the intervention device 30 exceeds a predetermined threshold, for example.

In an exemplary embodiment of the present invention, means may be provided to enable the automatic selection and/or adaptation of certain visualisation parameters, depending on the determined position of the medical intervention device 30 within the 3D ultrasound data set. For example, the tip position of the intervention device may be used for the definition of the intersection point of, for example, three, possibly (but not necessarily) orthogonal slices (or thin 3D slabs) cut out of the volume 120 defined by the ultrasound scanhead 10, as illustrated schematically in FIG. 5 of the drawings. Alternatively, the tip position could be used to define a cut plane 140, as illustrated schematically in FIG. 6 of the drawings, which cut plane 140 separates visualised volume information 142 from cut volume information 144. Of course, the cut volume portion 144 need not necessarily be suppressed, but could alternatively be shown in, say, side-by-side relation to the visualised volume information 142.

The orientation of the intervention device may be used to, for example, align a slice (or 3D slab) with the device and the shape of the intervention device may be used, for example, to perform a curved visualisation through the volume along the intervention device.

It is envisaged that the system of the present invention would be suitable in a number of different applications including biopsy procedures and a wide range of invasive procedures, such as the placement of stents and cannulae, the dilation or resection of vessels, treatments involving the freezing or heating of internal tissues, the placement of radioactive seeds or prosthetic devices such as valves and rings, the guidance of wires or catheters through vessels for the placement of devices such as pacemakers, implantable cardiovertors/defibrillators, electrodes and guide wires, the placement of sutures, staples and chemical/gene sensing electrodes, the guidance or operation of robotic surgical devices, and the guidance of endoscopic or minimally invasive surgical procedures. Ultrasonic (or other modality) guidance such as that provided by the present invention would thus find expanded use in a broad range of invasive or interventional clinical applications including cardiac, pulmonary, central and peripheral nervous system procedures, gastrointestinal, muskuloskeletal, gynaecological, obstetrical, urological, opthalmologic and otorhinolarygologic procedures, and the present invention is not necessarily intended to be limited in this regard.

It should be noted that the above-mentioned embodiments illustrate rather than limit the invention, and that those skilled in the art will be capable of designing many alternative embodiments without departing from the scope of the invention as defined by the appended claims. For example, the intervention device could be replaced by an anatomic landmark so as to enable visualisation and/or stabilisation of anatomical details such as heart valves over the motion cycle to be optimised.

In the claims, any reference signs placed in parentheses shall not be construed as limiting the claims. The word “comprising” and “comprises”, and the like, does not exclude the presence of elements or steps other than those listed in any claim or the specification as a whole. The singular reference of an element does not exclude the plural reference of such elements and vice-versa. The invention may be implemented by means of hardware comprising several distinct elements, and by means of a suitably programmed computer. In a device claim enumerating several means, several of these means may be embodied by one and the same item of hardware. The mere fact that certain measures are recited in mutually different dependent claims does not indicate that a combination of these measures cannot be used to advantage.

1. An imaging system for generating for display live, three-dimensional images of a body volume, the system comprising
two dimensional array transducer for scanning said body volume so as to obtain live three-dimensional images in respect of said body volume,
object recognition means for identifying, within one or more of said live three dimensional images of said body volume, the relative location of a selected object within said body volume,
means for selecting an imaging plane corresponding to said location of said object, and
means for generating a live three-dimensional image intersected by a delineation of said selected imaging plane containing the location of the object.

2. An ultrasonic imaging system according to claim 1, wherein said two dimensional array transducer is operable for generating incident beams in the body volume and for receiving beams reflected from or transmitted through said body volume.

3. An ultrasonic imaging system according to claim 2, wherein the two dimensional array transducer is further operable to electronically steer incident beams to scan the selected imaging plane including the location of the object.

4. (canceled)

5. An ultrasonic imaging system according to claim 1, wherein the object recognition means is further operable to identify the location of said selected object by segmenting or filtering said live images to enhance the appearance therein of said selected object, and then defining the location of the object within the body volume by one or more reference points relative to at least a portion of the object.

6. An ultrasonic imaging system according to claim 1, wherein the object recognition means further comprises means for determining the orientation of the object relative to the body volume.

7. An ultrasonic imaging system according to claim 1, wherein said object is a medical intervention device.

8. (canceled)

9. An ultrasonic imaging system according to claim 1, wherein said live, three-dimensional images of said body volume comprise three-dimensional ultrasound images.

10. An ultrasonic imaging system according to claim 1, wherein said live, three-dimensional images are obtained, and the location of said selected object therein determined, substantially simultaneously.
